SEQUENCE LISTING (24 sequences)

SEQ ID NO: 1 - DNA, 729 bp, Oryza sativa
  CDS (1)..(729)
atggcaatggaggctgcggcggagaggagcgccggagcaggggcggcggcgacggcggcgccggagagcggtggcggtggtgcaggggagaggcggtcgcggttcaggcggatctgcgtgtactgcggcagcgccaaggggaggaaggccagctaccaggacgccgccgtcgagctcggcaaggaactggtcgagaggggcatagacctggtctacggcggtggctccatcggcctcatgggcctcgtctcccacgctgttcacgacggtggtcgccatgtcattggggtcatcccgaaatccttgatgcccagagaggtcactggtgagcctgttggtgaagttagagcggtctctggcatgcacgagaggaaggctgaaatggcccggtttgctgatgcgttcattgcactgcctggcggctacgggactcttgaggagttgcttgaggtcatcacctgggcccaactaggaatccacaagaagccggttggccttctgaatgtcgacgggttttatgatccttttctatccttcattgacatggctgtcagcgaaggattcatagcggaggatgcgcggcgcattatcatctcggctccaactgccagggagctagttctgaagcttgaggagtatgttcccgagtacgaggtcggcttggtttgggacgatcagatgccgcacagcttcgcgcctgacctcgagaccaggatcacctcatcctga

SEQ ID NO: 2 - protein, 242 aa, Oryza sativa
MAMEAAAERSAGAGAAATAAPESGGGGAGERRSRFRRICVYCGSAKGRKASYQDAAVELGKELVERGIDLVYGGGSIGLMGLVSHAVHDGGRHVIGVIPKSLMPREVTGEPVGEVRAVSGMHERKAEMARFADAFIALPGGYGTLEELLEVITWAQLGIHKKPVGLLNVDGFYDPFLSFIDMAVSEGFIAEDARRIIISAPTARELVLKLEEYVPEYEVGLVWDDQMPHSFAPDLETRITSS

SEQ ID NO: 3 - DNA, 642 bp, arabidopsis
  CDS (1)..(642)
atggagatagaatcaaagttcaagagaatatgtgtgttttgtggaagtagtgctggtaataaagtcagttacaaagatgctgctatcgaactcggaaccgaactggtatcgagaaatatcgatcttgtctatggtggagggagcattggtttaatgggtttgatttctcaagctgttttcaatggaggtcgccatgtcattggggttatccccaagacactaatgccaagagagataacaggagagacagtgggagaagtgaaggcagtagcagacatgcaccaaaggaaagctgaaatggctaagcactctgatgcttttatcgctttacctggtgggtatgggacacttgaagagcttcttgaagttatcacttgggcacagctaggcatccatgataaaccagtgggactgttgaatgtggaaggatactacaattcattgttatcattcatagacaaagcagtggaagaaggtttcatttcaccaactgctcgtcatatcattgtatctgctccttctgctaaagaacttgtcaagaaacttgaggattatgtaccaaggcatgagaaggtagcctcaaagaaaagctgggagatggagcaaatagggctaagtcccacttgtgaaatctcaagatga

SEQ ID NO: 4 - protein, 213 aa, arabidopsis
MEIESKFKRICVFCGSSAGNKVSYKDAAIELGTELVSRNIDLVYGGGSIGLMGLISQAVFNGGRHVIGVIPKTLMPREITGETVGEVKAVADMHQRKAEMAKHSDAFIALPGGYGTLEELLEVITWAQLGIHDKPVGLLNVEGYYNSLLSFIDKAVEEGFISPTARHIIVSAPSAKELVKKLEDYVPRHEKVASKKSWEMEQIGLSPTCEISR

SEQ ID NO: 5 - DNA, 642 bp, arabidopsis
  CDS (1)..(642)
  CDS (1)..(642)
atggaagagacaaaatcaagattcaggaggatctgtgtcttctgcggaagcagttccggcaacaaaaccacttatcacgacgctgctctccaactcgcccaccaactggtggagagaaacatcgatttggtgtatggaggaggaagcgtggggctaatgggtctcatttctcaagctgttcatgatggtggtcgtcatgttcttgggatcattccgaagtctctagcaccaagagagataaccggtgaatcaataggagaagttataacagtatcgactatgcaccaaaggaaggctgaaatgggtcgccaagcggatgccttcatcgcacttcctggtggatatgggacatttgaagagttgttggaagtcatcacctggtctcagcttgggattcacactaaaccggtgggactattgaacgtggatggattctacgattcactattgacgtttatagataaggcagtggacgaaggcttcgtctcctccaccgctcgccgtatcattgtctcggctccaaacgctcctcaattacttcaacttcttgaggagtatgttccaaaacacgacgattttgtatccaaaatggtgtgggacaataccacggatgctttcactttggaaggcgactcgttttag

SEQ ID NO: 6 - protein, 213 aa, arabidopsis
MEETKSRFRRICVFCGSSSGNKTTYHDAALQLAHQLVERNIDLVYGGGSVGLMGLISQAVHDGGRHVLGIIPKSLAPREITGESIGEVITVSTMHQRKAEMGRQADAFIALPGGYGTFEELLEVITWSQLGIHTKPVGLLNVDGFYDSLLTFIDKAVDEGFVSSTARRIIVSAPNAPQLLQLLEEYVPKHDDFVSKMVWDNTTDAFTLEGDSF

SEQ ID NO: 7 - DNA, 648 bp, arabidopsis
  CDS (1)..(648)
atggaaatcaaaggtgaatcgatgcaaaagtcaaagttcagaagaatctgtgtcttctgtggaagcagccaaggcaagaagagcagttaccaagatgctgctgttgacctcggcaacgaactggtttcaaggaatattgatctagtctatggaggtgggagcataggattgatgggtttggtttcacaagctgttcatgatggtggtcgtcatgttattggaatcattcccaagaccctcatgcctagagagttgactggtgaaacagtaggagaagtaagagcagttgcagatatgcaccaaaggaaagctgagatggctaagcactctgatgcttttattgccttaccaggtggttatggaacacttgaagaattgcttgaagtcataacttgggctcagcttggtatacatgacaagccggtgggtttgctcaatgttgatggatactacaactctctgctctcattcattgacaaagcagtcgaagaaggatttattagcccgactgctcgtgagatcatcgtctccgcacctactgctaaagagctggtgaaaaagctagaggaatatgcaccttgccatgaaagggttgcaacgaagctttgttgggagatggaacggattggttactcctctgaagagtga

SEQ ID NO: 8 - protein, 215 aa, arabidopsis
MEIKGESMQKSKFRRICVFCGSSQGKKSSYQDAAVDLGNELVSRNIDLVYGGGSIGLMGLVSQAVHDGGRHVIGIIPKTLMPRELTGETVGEVRAVADMHQRKAEMAKHSDAFIALPGGYGTLEELLEVITWAQLGIHDKPVGLLNVDGYYNSLLSFIDKAVEEGFISPTAREIIVSAPTAKELVKKLEEYAPCHERVATKLCWEMERIGYSSEE

SEQ ID NO: 9 - DNA, 648 bp, arabidopsis
  CDS (1)..(648)
atggaggtcaacaatgaaaccatgcaaaagtcaaagtttggaagaatctgtgtgttttgtggaagcagccaaggcaagaagagtagttaccaagatgctgctgtggatctaggcaacgaattggttttaaggaacattgatctagtctatggaggtggaagcataggtttgatgggtttggtttcgcaagctgttcatgatggtggtcgccatgttattggagttattcccaagacactcatgcctagagagttgaccggtgaaacagtaggagaagtaagagcagttgcagatatgcatcaaagaaaagcagagatggctagacactctgatgcttttattgctttaccaggtggatatggaacacttgaagagcttttggaggtcataacatgggctcagcttggaatacatgataaaccggtgggtttgctcaatgttgatggatactacaactctttgctctctttcattgataaagccgttgaagaaggcttcatcagtacaaacgcacgccagatcataatttctgcacctactgccaaggagcttgtaaagaagctggaggaatattcgccttgccatgaaagtgttgcgactaagctttgttgggagatagagcggattgactactcttctgaagactga

SEQ ID NO: 10 - protein, 215 aa, arabidopsis
MEVNNETMQKSKFGRICVFCGSSQGKKSSYQDAAVDLGNELVLRNIDLVYGGGSIGLMGLVSQAVHDGGRHVIGVIPKTLMPRELTGETVGEVRAVADMHQRKAEMARHSDAFIALPGGYGTLEELLEVITWAQLGIHDKPVGLLNVDGYYNSLLSFIDKAVEEGFISTNARQIIISAPTAKELVKKLEEYSPCHESVATKLCWEIERIDYSSED

SEQ ID NO: 11 - DNA, 687 bp, arabidopsis
  CDS (1)..(687)
atggaaatagtgaagtcgaggttcaagagggtttgtgtgttctgtggtagcagcagcggaaagagagagtgctacagtgatgccgccactgatctagctcaagagctggtgacgaggagattgaatcttgtgtatggaggaggaagcattggtctcatgggtttggtctcacaagctgttcatgaagctggaggacatgtactagggatcataccaaggactcttatggacaaagagataaccggagaaacatatggtgaggtaatagctgtggcggatatgcatgaaagaaaagccgaaatggcacgccactcggattgtttcattgctttaccaggtgggtatggaacactggaggagttattggaagtaatagcatgggcacaacttggaattcacgacaagcctgtgggtttgttaaatgtggatggttattacaattacctcctcactttcattgataaagccgttgatgatggctttatcaaaccatctcagcgtcacatctttgtctcagcccccaatgccaaagagcttgtccaaaaacttgaggcatacaagccagtgaatgatggagtcatagctaaatctaggtgggaggttgagaagaaagtgcaacagccgcaacaacagcaacaagtagtgttctgttctaacacaagcatgcagactgagattgccctttag

SEQ ID NO: 12 - protein, 228 aa, arabidopsis
MEIVKSRFKRVCVFCGSSSGKRECYSDAATDLAQELVTRRLNLVYGGGSIGLMGLVSQAVHEAGGHVLGIIPRTLMDKEITGETYGEVIAVADMHERKAEMARHSDCFIALPGGYGTLEELLEVIAWAQLGIHDKPVGLLNVDGYYNYLLTFIDKAVDDGFIKPSQRHIFVSAPNAKELVQKLEAYKPVNDGVIAKSRWEVEKKVQQPQQQQQVVFCSNTSMQTEIAL

SEQ ID NO: 13 - DNA, 654 bp, arabidopsis
  CDS (1)..(654)
atggaagagacaaaatcgagattcaagaggatctgtgtcttctgtggaagcagttccggcaaaaaaccttcataccaagaagctgccattcaattgggtaacgagttggtggagagaaggattgatttggtatacggaggtggtagcgtggggcttatgggtctcgtctctcaagctgttcatcatggtggtcgccatgttctaggggtcattccaaaaaccttgatgccaagagagataactggtgagaccatcggagaagttaaagccgtggccgatatgcatcaaaggaaagctgaaatggctcgccaagccgacgcattcattgcccttcctggtgggtatggtacgttagaagaattgctggaagtcattacatgggctcaactcggtatccaccgtaagccggtgggtcttcttaacgtggatggttactacaactcgctgttaacgtttattgataaggctgtggacgaaggatttatatccccaatggctcgtcgaatcatcgtctcagctccaaacgctaaagagttggttcgacaactcgaggaatatgaaccggagtttgatgagataacatcaaaattggtttgggatgaagtggaccggataagttatgtaccgggttcggaggtagctaccgctacgtaa

SEQ ID NO: 14 - protein, 217 aa, arabidopsis
MEETKSRFKRICVFCGSSSGKKPSYQEAAIQLGNELVERRIDLVYGGGSVGLMGLVSQAVHHGGRHVLGVIPKTLMPREITGETIGEVKAVADMHQRKAEMARQADAFIALPGGYGTLEELLEVITWAQLGIHRKPVGLLNVDGYYNSLLTFIDKAVDEGFISPMARRIIVSAPNAKELVRQLEEYEPEFDEITSKLVWDEVDRISYVPGSEVATAT

SEQ ID NO: 15 - DNA, 651 bp, arabidopsis
  CDS (1)..(651)
atggaagataatcagcgaagcagattcagaaaaatctgtgtcttttgcggaagccactctggtcacagagaagttttcagtgatgctgccatcgaactcggcaatgaactcgtgaagaggaagatagatttggtttatggaggaggaagtgttggattgatgggtttgatatccaggagagtctatgaaggtggtttacatgtacttggaatcattcccaaagctttgatgccaattgagatatctggtgagactgtgggagatgtaagagttgttgcagacatgcatgagcgaaaggctgcaatggcacaggaagctgaggccttcattgcactccctggaggttatggaacaatggaggagctgttggagatgataacatggtcacaacttggtatccataagaagacggttggtctattgaatgttgatgggtactataacaatttgcttgctttatttgataccggtgtcgaagaaggttttatcaagccaggtgcacgtaacattgtggtttctgctccaacagccaaagagcttatggagaagatggaggaatatactccttcacacatgcatgttgcatcgcacgaaagctggaaagttgaagaactcggagattacccgggacaagaaaacaagccgcaataa

SEQ ID NO: 16 - protein, 216 aa, arabidopsis
MEDNQRSRFRKICVFCGSHSGHREVFSDAAIELGNELVKRKIDLVYGGGSVGLMGLISRRVYEGGLHVLGIIPKALMPIEISGETVGDVRVVADMHERKAAMAQEAEAFIALPGGYGTMEELLEMITWSQLGIHKKTVGLLNVDGYYNNLLALFDTGVEEGFIKPGARNIVVSAPTAKELMEKMEEYTPSHMHVASHESWKVEELGDYPGQENKPQ

SEQ ID NO: 17 - DNA, 1557 bp, Arabidopsis thaliana
  CDS (1)..(1557)
atgttgcttactatattaaaatcactcctcgtgatattcgtgactacgatattgagagttttatacgacaccatatcgtgctactggctaacacctagacgaatcaagaagatcatggaacagcaaggcgtaacgggtcctaaaccgcgtccgttaaccggaaatatccttgaaatctcggccatggtttcgcaatccgcttccaaggattgtgactctattcaccatgacatcgtcggccgccttctcccgcattacgttgcttggtccaaacaatacgggaagaggtttattgtgtggaacgggacggatcctcggctttgcttaacggaaacggaattgataaaggagttgctgatgaaacacaacggtgttagtggaagatcgtggctacaacaacaagggactaagaattttatcggtcgcggtctcctaatggctaatggccaagattggcaccaccaacgccaccttgctgcaccggcatttaccggagaaagactcaagggatacgcaaggcatatggtggagtgcacaagcaagctagttgagaggttgaggaaggaagttggggaaggagcaaatgaggtggagataggagaggagatgcataaactcacggctgatattatatcgaggacaaagttcggaagcagctttgagaaaggcaaagagcttttcaaccacctcactgtcctccagcgccgttgcgctcaagccacccgccacctctgctttcccggcagccggtttcttccaagcaaatacaacagagagatcaagtctctgaaaaaagaagttgagcgtttgttgattgagatcatacaaagcaggcgagactgcgctgaaatgggtaggagtagcactcacggtgacgaccttctcgggcttctcttgaacgaaatggatattgacaagaacaataacaataacaacaacaatcttcagttgataatggatgaatgcaagacattcttctttgctggccatgaaaccacggctttgctcctcacatggacaacaatgctcctcgccgataatcccacatggcaggaaaaagtccgtgaggaggtcagggaggtctttggccgcaatggccttccctcagtcgatcaactatctaagctaacctcgttaagcaaagtgattaacgagtcactaagactttaccctccagctactcttctaccaaggatggcatttgaagatctaaaactaggtgatctaaccattcctaaaggtttatcaatatggataccggttcttgctatccatcacagtgaagaattatggggtaaagacgcgaaccaattcaatcccgaacgttttggaggcagaccattcgcgtctggtcgccatttcatcccatttgcagccggtccacggaactgcatcggacaacagtttgcgttgatggaagctaaaataatattagccacgttaatttcaaagtttaacttcacaatatcaaagaattataggcatgccccaatcgttgtgcttactataaaacctaagtatggagttcaagtgatattgaagccattggtttcatga

SEQ ID NO: 18 - protein, 518 aa, Arabidopsis thaliana
MLLTILKSLLVIFVTTILRVLYDTISCYWLTPRRIKKIMEQQGVTGPKPRPLTGNILEISAMVSQSASKDCDSIHHDIVGRLLPHYVAWSKQYGKRFIVWNGTDPRLCLTETELIKELLMKHNGVSGRSWLQQQGTKNFIGRGLLMANGQDWHHQRHLAAPAFTGERLKGYARHMVECTSKLVERLRKEVGEGANEVEIGEEMHKLTADIISRTKFGSSFEKGKELFNHLTVLQRRCAQATRHLCFPGSRFLPSKYNREIKSLKKEVERLLIEIIQSRRDCAEMGRSSTHGDDLLGLLLNEMDIDKNNNNNNNNLQLIMDECKTFFFAGHETTALLLTWTTMLLADNPTWQEKVREEVREVFGRNGLPSVDQLSKLTSLSKVINESLRLYPPATLLPRMAFEDLKLGDLTIPKGLSIWIPVLAIHHSEELWGKDANQFNPERFGGRPFASGRHFIPFAAGPRNCIGQQFALMEAKIILATLISKFNFTISKNYRHAPIVVLTIKPKYGVQVILKPLVS

SEQ ID NO: 19 - DNA, 1539 bp, Arabidopsis thaliana
  CDS (1)..(1539)
atgatggttacattagtactaaagtacgtcctcgtgatagtcatgaccttgatattaagagtcctatacgactccatttgttgctacttcctcacaccaagacgaatcaagaagtttatggaacgtcaaggcatcacggggcctaaaccccgtctcctcaccggaaacatcattgatatctccaaaatgctctctcactccgcttctaacgactgctcttccatccaccacaacatcgtcccacgccttcttcctcattacgtctcttggtccaaacaatatgggaagagatttataatgtggaacgggacagaaccgagactatgcctaacggagaccgagatgattaaagaattgctaacgaagcacaatccggtcaccgggaaatcatggctgcaacaacaaggaactaaagggtttatagggcgtgggcttctcatggccaacggcgaggcttggcaccaccaacgtcacatggcagctccggctttcacacgtgataggctcaaaggatacgctaagcatatggtggaatgcacgaagatgatggctgagaggctgagaaaggaggttggagaagaggtggaaattggtgaggagatgcggcggctcacggcggatataatatcgaggacggaatttggaagtagttgtgataaaggaaaggagctttttagtctactcactgtacttcaacgtctttgtgctcaagccactcgccacctctgttttcccggtagccggtttctacctagcaaatacaatagagaaataaagtctctgaaaacggaagtggaacgtcttttgatggagattatagacagccgaaaagacagtgtagagatcggtcggagtagctcatacggggatgacctcttagggcttcttttgaaccagatggatagcaacaagaacaaccttaatgttcaaatgataatggatgagtgcaagactttcttctttaccggtcatgagacaacctctcttctcctcacgtggactctcatgctccttgctcataatcccacttggcaggacaatgtccgcgatgaggtccggcaagtttgtggccaagatggtgtcccttccgttgaacagctctcaagtcttacttcgttaaacaaagtgataaacgagtcattaagactttaccctcctgccacacttttaccaagaatggcatttgaagacataaaactaggtgacctaatcatccctaaaggtttatctatttggatccctgtccttgcgatccatcatagtaatgaattatggggcgaagatgcgaacgagttcaaccctgaacgatttactactagaagttttgcatctagtcgccattttatgccttttgcggctggacctaggaactgcattggccagacttttgccatgatggaggcgaagataatattagcaatgcttgtttccaaattcagttttgcaatatcggagaactatagacatgctcctatagttgtgcttactataaaacctaagtatggagttcaattagttttgaagccacttgatctatga

SEQ ID NO: 20 - protein, 512 aa, Arabidopsis thaliana
MMVTLVLKYVLVIVMTLILRVLYDSICCYFLTPRRIKKFMERQGITGPKPRLLTGNIIDISKMLSHSASNDCSSIHHNIVPRLLPHYVSWSKQYGKRFIMWNGTEPRLCLTETEMIKELLTKHNPVTGKSWLQQQGTKGFIGRGLLMANGEAWHHQRHMAAPAFTRDRLKGYAKHMVECTKMMAERLRKEVGEEVEIGEEMRRLTADIISRTEFGSSCDKGKELFSLLTVLQRLCAQATRHLCFPGSRFLPSKYNREIKSLKTEVERLLMEIIDSRKDSVEIGRSSSYGDDLLGLLLNQMDSNKNNLNVQMIMDECKTFFFTGHETTSLLLTWTLMLLAHNPTWQDNVRDEVRQVCGQDGVPSVEQLSSLTSLNKVINESLRLYPPATLLPRMAFEDIKLGDLIIPKGLSIWIPVLAIHHSNELWGEDANEFNPERFTTRSFASSRHFMPFAAGPRNCIGQTFAMMEAKIILAMLVSKFSFAISENYRHAPIVVLTIKPKYGVQLVLKPLDL

SEQ ID NO: 21 - DNA, 1605 bp, Oryza sativa
  CDS (1)..(1605)
atggccgccgccgtcctcgtcgccatcgcattgccggtctcgctggcgctgctgctggtggccaaggccgtctgggtcaccgtctcatgctactacctcacgccggcgaggatccggcgggtcctggcgagccagggcgtgcgcggcccgccgccgcggccgctcgtcggcaacctccgcgacgtgtcggcgctcgtcgccgagtccaccgccgccgacatggcctccctcagccacgacatcgtcgcccgcctcctcccccactacgtcctctggtccaacacgtacgggaggcggttcgtgtactggtacgggagcgagccgcgggtgtgcgtgacggaggccggcatggtgcgggagctcctgtcgtcgcggcacgcgcacgtcaccggcaagtcgtggctgcagcggcagggcgccaagcacttcatcggccgtggcctcctcatggccaacggcgccacctggtcgcaccagcgccacgtcgtcgcgccggcgttcatggccgaccggctcaaggggagggtggggcacatggtggagtgcacgaggcagacggtgcgggcgctgagggatgcggtggcgaggtccgggaacgaggtggagatcggcgcgcacatggcgaggctcgccggcgacgtgatcgcgcgcaccgagttcgacacgagctacgagaccggcaagaggatcttcctcctcatcgaggagctccagcgcctcaccgcccgctccagccgctacctctgggtccccggcagccagtattttccgagcaagtacaggagagagataaagcggctgaacggcgagctggagcggctgctcaaggagtccatcgaccggagccgggagatcgccgacgagggccggacgccgtcggcgtcgccgtgcggccgtggcctcctcggcatgctgctggccgagatggagaagaaggaggccggcggcaatggcggcggcgaggtcgggtacgacgcccagatgatgatcgacgagtgcaagaccttcttcttcgccggccacgagacgtcggcgctgctcctcacctgggccatcatgctgctcgccacgcacccggcgtggcaggacaaggcgcgcgccgaggtcgccgccgtctgcggcggcggcgcgccgtcgccggacagcctcccgaagctcgccgtgctccagatggtgatcaacgagacgctgcggctgtacccgccggcgacgctgctgccgcggatggcgttcgaggacatcgagctcggcgggggcgcgctccgggtgccgagtggcgcgtcggtgtggatcccggtgctcgccatccaccacgacgagggcgcgtggggccgcgacgcgcacgagttcaggccggacaggttcgcgccgggacggccgcggccgccggcgggggcgttcctgccgttcgccgccgggccgcgcaactgcgtcgggcaggcgtacgccatggtggaggccaaggtcgcgctcgccatgctcctctccagcttccgcttcgccatctccgacgagtaccggcacgcgccggtgaacgtgctcacgctccggccacgccacggcgtgcccgtccgcctcctgccgctgccgccgccgcgcccatag

SEQ ID NO: 22 - protein, 534 aa, Oryza sativa
MAAAVLVAIALPVSLALLLVAKAVWVTVSCYYLTPARIRRVLASQGVRGPPPRPLVGNLRDVSALVAESTAADMASLSHDIVARLLPHYVLWSNTYGRRFVYWYGSEPRVCVTEAGMVRELLSSRHAHVTGKSWLQRQGAKHFIGRGLLMANGATWSHQRHVVAPAFMADRLKGRVGHMVECTRQTVRALRDAVARSGNEVEIGAHMARLAGDVIARTEFDTSYETGKRIFLLIEELQRLTARSSRYLWVPGSQYFPSKYRREIKRLNGELERLLKESIDRSREIADEGRTPSASPCGRGLLGMLLAEMEKKEAGGNGGGEVGYDAQMMIDECKTFFFAGHETSALLLTWAIMLLATHPAWQDKARAEVAAVCGGGAPSPDSLPKLAVLQMVINETLRLYPPATLLPRMAFEDIELGGGALRVPSGASVWIPVLAIHHDEGAWGRDAHEFRPDRFAPGRPRPPAGAFLPFAAGPRNCVGQAYAMVEAKVALAMLLSSFRFAISDEYRHAPVNVLTLRPRHGVPVRLLPLPPPRP

SEQ ID NO: 23 - DNA, 1608 bp, Oryza sativa
  CDS (1)..(1608)
atggcggtcctcgtgtcgctcatggtgatcgccgcgtcgtcgcctctcgtggcgctgcttctgagggcggcgtgggtgaccctgtcctgctactggctgacgccaatgaggatccgccgcgccatggcggcgcagggcgtgcgcggcccgccgccgcgcccgctcgtcggcaacctccgggaggtgtcggcgctcgtggcgagggccaccgccgacgacatgccgtccctcagccacgacatcgtcggccgcctcatgccccactacgtgctctggtccgggacatacggcaagctgttcgtgtacctgtacgggagcgagccgaggctgtgcctgactgacaccgcgctgatcaaggagttcttgtcgtccaagtacgcccacgccaccggcaagtcgtggctgcagaggcaggggacgaagcacttcatcggcggcgggctgctcatggccaacggcgccaggtgggcgcaccagcgccacgtcgtcgcgccggcgttcatggccgacaagctcaaggcacgcgggagagtcgggcgcatggtggagtgcacgaagcaggcgatccgcgagctccgcgacgcggcggcggggcggcgcggcgaggaggtggagatcggcgcccacatgacccgcctcaccggcgacatcatctcccgcaccgagttcaacaccagctacgacactggcaagcgcatcttcctcctcctcgagcacctccagcgcctcacctcccgctccagccgccatctctggatccccggcagccagtatttcccgagcaagtacaggagggagatcaggcggctcaacggcgagctggaggcggtgctgatggagtcgatacggcggagcagggagatcgccgacgaggggagggcggcggtggcgacgtacgggaggggtttgctggctatgcttctgtcggagatggaggagaaggagaagaatggcggcggcggaggcggcgagttcagctacgacgcgcagctggtgatcgacgagtgcaagaccttcttcttcgccggccacgagacgtcggcgctgctgctcacgtgggcgatcatgctgctcgccaccaacccggcgtggcaggagaaggcccgcaccgaggtcgccgccgtctgcggcgaccacccgccgtccgccgaccacctctccaagctcaccgtgctgcagatgatcatccaggagacgctgcggctgtacccgccggcgacgctgctgccgcggatggcgttcgaggacatccagctcggcggcctccggctgccgcgggggctgtcggtgtggatcccggtgctggccatccaccacgacgagtccatctggggccccgacgcgcacgagttccgcccggagaggttcgcgccgggcgcgcgccgcccgtcggccgccggcgccgccaggttcctgccgttcgccgccggcccgcgcaactgcgtcggccaggcgtacgcgctcgtcgaggccaaggtcgtcctcgccatgctcctctcggccttccgcttcgccatctccgacaactaccgccacgcgccggagaacgtgctcaccctccgccccaagcacggcgtccccgtccacctccggccgctgcggccatag

SEQ ID NO: 24 - protein, 535 aa, Oryza sativa
MAVLVSLMVIAASSPLVALLLRAAWVTLSCYWLTPMRIRRAMAAQGVRGPPPRPLVGNLREVSALVARATADDMPSLSHDIVGRLMPHYVLWSGTYGKLFVYLYGSEPRLCLTDTALIKEFLSSKYAHATGKSWLQRQGTKHFIGGGLLMANGARWAHQRHVVAPAFMADKLKARGRVGRMVECTKQAIRELRDAAAGRRGEEVEIGAHMTRLTGDIISRTEFNTSYDTGKRIFLLLEHLQRLTSRSSRHLWIPGSQYFPSKYRREIRRLNGELEAVLMESIRRSREIADEGRAAVATYGRGLLAMLLSEMEEKEKNGGGGGGEFSYDAQLVIDECKTFFFAGHETSALLLTWAIMLLATNPAWQEKARTEVAAVCGDHPPSADHLSKLTVLQMIIQETLRLYPPATLLPRMAFEDIQLGGLRLPRGLSVWIPVLAIHHDESIWGPDAHEFRPERFAPGARRPSAAGAARFLPFAAGPRNCVGQAYALVEAKVVLAMLLSAFRFAISDNYRHAPENVLTLRPKHGVPVHLRPLRP

(19) United States
(12) Patent Application Publication
(10) Pub. No.: US 2013/0212743 A1
(43) Pub. Date: Aug. 15, 2013

(54) METHOD FOR INCREASING PLANT BIOMASS BY CONTROLLING ACTIVE CYTOKININ EXPRESSION LEVEL

(71) Applicants: Takeshi Kuroha, Yokohama-shi (JP); Hitoshi Sakakibara, Yokohama-shi (JP)

(72) Inventors: Takeshi Kuroha, Yokohama-shi (JP); Hitoshi Sakakibara, Yokohama-shi (JP)

(73) Assignee: RIKEN, Saitama (JP)

(21) Appl. No.: 13/878,737

(22) PCT Filed: Oct. 26, 2011
(86) PCT No.: PCT/JP2011/075237

§ 371 Date: Apr. 10, 2013

(30) Foreign Application Priority Data
Oct. 27, 2010 (JP) 2010-241241

(51) Int. Cl. C12N 15/82

(52) U.S. Cl. CPC C12N 15/8243
USPC 800/290; 800/298

(57) ABSTRACT

An object of the present invention is to provide a means for conferring a useful phenotype to a plant by efficiently controlling the quantitative productivity of iP-type and tZ-type active cytokinins in plant aerial organs. According to the present invention, a method for increasing plant biomass by causing the overexpression of an active cytokinin synthase gene and a cytokinin hydroxylase gene in a plant body is provided.

### TECHNICAL FIELD

The present invention relates to a method for increasing plant biomass by controlling the expression levels of active cytokinins, a transgenic plant with increased plant biomass, and a method for producing the transgenic plant.

### BACKGROUND ART

Cytokinins play multidimensional roles in various aspects of plant development and growth such as control of cell division activity, leaf aging, root formation, apical bud dominance, and bud dormancy. Cytokinins are plant hormones that are extremely important for controlling the quantitative productivity of crops.

Based on the findings from conventional studies concerning cytokinin metabolic pathways, it has been revealed that in a plant body, active cytokinin (base form) is synthesized by the following reaction pathways. First, in the first reaction of cytokinin synthesis, nucleotide cytokinin is produced as a result of a condensation reaction between adenine nucleotide and dimethylallyl diphosphate (DMAPP). The nucleotide form does not have activity as cytokinin in a plant body. In the second reaction, such nucleotide form is converted to an active base form as a result of dissociation of ribose phosphate. The following two types of pathway are thought to be involved in this process: a two-stage pathway, by which a nucleotide form is converted to a nucleoside form through dissociation of a phosphate group, followed by conversion of the nucleoside form to a base form as a result of dissociation of ribose; and a direct pathway (one-stage pathway), by which ribose phosphate is directly dissociated through the mediation of enzyme genes (LOG) (discovered by the present inventors) that catalyze cytokinin activation reactions (Non-patent Literature 1).

The present inventors have reported that the LOG genes are required to maintain the shoot apex meristem (division) activity of rice aerial parts (Non-patent Literature 2), and that a group of AtLOG genes that are LOG homolog genes of Arabidopsis thaliana is required for the development and the growth of plant bodies in which cytokinins are involved (Non-patent Literature 3).

There are various types of cytokinin of plant bodies, depending on differences in adenine side chains. Major cytokinins are of the tZ (trans-zeatin) type, the cZ (cis-zeatin) type, and the iP (isopentenyladenine (iP)) type (Non-patent Literature 1).

Enzyme genes (LOG) catalyzing cytokinin activation reactions can synthesize active cytokinins from nucleotide cytokinins in one stage as described above, so that active cytokinins can be directly controlled quantitatively. However, overexpression of AtLOG genes in Arabidopsis thaliana resulted in an effect such that the amount of iP-type active cytokinin was increased in aerial part organs, while conversely, the amount of the tZ-type active cytokinin was decreased (Patent Literature 1, Non-patent Literature 3). Meanwhile, based on the research conducted by the present inventors, it has been revealed that the tZ-type cytokinin is more important than the iP-type cytokinin in maintenance of shoot apex meristem activity of aerial parts, in which cytokinins are involved (Patent Literature 2). Reflecting this, delayed leaf aging or development of leaf vascular bundles was observed in AtLOG gene overexpressing plants, while downsizing of plant bodies was also observed (Patent Literature 1, Non-patent Literature 3).

(Patent Literature 1) WO2008/029942: “Use of active cytokinin synthase gene”
(Patent Literature 2) JP Patent Publication (Kokai) No. 2006-314206 A: “Method for producing dwarfing plants or plants with many flower stalks”
(Non-patent Literature 1) Sakakibara, H. (2006) Cytokinins: Activity, biosynthesis and translocation. Annu. Rev. Plant Biol. 57: 431-449.
(Non-patent Literature 2) Kurakawa, T., Ueda, N., Maekawa, M., Kobayashi, K., Kojima, M., Nagato, Y., Sakakibara, H., and Kyozuka, J. (2007). Direct control of shoot meristem activity by a cytokinin-activating enzyme. Nature 445: 652-655.
(Non-patent Literature 3) Kuroha, T., Tokunaga, H., Kojima, M., Ueda, N., Ishida, T., Nagawa, S., Fukuda, H., Sugimoto, K., Sakakibara, H. (2009) Functional Analyses of the LONELY GUY Cytokinin-Activating Enzymes Reveal the Importance of the Direct Activation Pathway in Arabidopsis. Plant Cell 21: 3152-3169.

### DISCLOSURE OF INVENTION

An object of the present invention is to provide a means for conferring a useful phenotype upon a plant by controlling the quantitative productivity of iP-type and tZ-type active cytokinins in plant aerial organs.

As a result of intensive studies to achieve the above object, the present inventors have found that the amount of active tZ-type cytokinin is decreased with the overexpression of the AtLOG gene in Arabidopsis thaliana as described above, since the iP-type cytokinin nucleotide is synthesized prior to the tZ-type cytokinin nucleotide in the cytokinin synthetic pathway, and most iP-type cytokinin nucleotides to be used for tZ-type cytokinin synthesis are already converted to active cytokinins. Furthermore, the present inventors have succeeded in increasing the amount of active tZ-type cytokinin in aerial parts by overexpressing the enzyme gene (CYP735A) that catalyzes conversion from an iP-type cytokinin to a tZ-type cytokinin together with the active cytokinin synthase gene (AtLOG) in Arabidopsis thaliana. The present inventors have further found that a new phenotype of increasing plant body size enormously can be conferred by co-overexpression of CYP735A and AtLOG, which was not possible to achieve with the overexpression of the LOG genes alone. The present invention has been completed based on these findings.

Specifically, the present invention includes the following inventions.
[1] A method for producing a transgenic plant with increased biomass, comprising introducing an active cytokinin synthase gene and a cytokinin hydroxylase gene into a plant cell so that they can be co-expressed, and regenerating a plant body from the plant cell.
[2] The method of [1], wherein the active cytokinin synthase gene is a LOG gene.
[3] The method of [1], wherein the cytokinin hydroxylase gene is a CYP735A gene.
[4] The method of [1], wherein the active cytokinin synthase gene is any one of the following genes (a) to (f):
   (a) a gene comprising DNA that consists of the nucleotide sequence shown in SEQ ID NO: 1, 3, 5, 7, 9, 11, 13, or 15;
   (b) a gene comprising DNA that hybridizes under stringent conditions to DNA consisting of a nucleotide sequence complementary to DNA consisting of the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
   (c) a gene comprising DNA that consists of a nucleotide sequence having 70% or higher identity with the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
   (d) a gene encoding a protein that consists of the amino acid sequence shown in SEQ ID NO: 2, 4, 6, 8, 10, 12, 14, or 16;
   (e) a gene encoding a protein that consists of an amino acid sequence derived from the amino acid sequence shown in SEQ ID NO: 2, 4, 6, 8, 10, 12, 14 or 16 by deletion, substitution, or addition of 1 or several amino acids, and has activity to catalyze a reaction for synthesizing an active cytokinin from a nucleotide cytokinin; and
   (f) a gene encoding a protein that consists of an amino acid sequence having 70% or higher identity with the amino acid sequence shown in SEQ ID NO: 2, 4, 6, 8, 10, 12, 14, or 16, and has activity to catalyze a reaction for synthesizing an active cytokinin from a nucleotide cytokinin.
[5] The method of [1], wherein the cytokinin hydroxylase gene is any one of the following genes (g) to (l):
   (g) a gene comprising DNA that consists of the nucleotide sequence shown in SEQ ID NO: 17, 19, 21, or 23;
   (h) a gene comprising DNA that hybridizes under stringent conditions to DNA consisting of a nucleotide sequence complementary to DNA consisting of the nucleotide sequence shown in SEQ ID NO: 17, 19, 21, or 23, and encodes a protein having activity to catalyze conversion from an iP-type cytokinin to a tZ-type cytokinin;
   (i) a gene comprising DNA that consists of a nucleotide sequence having 70% or higher identity with the nucleotide sequence shown in SEQ ID NO: 17, 19, 21, or 23, and encodes a protein having activity to catalyze conversion from an iP-type cytokinin to a tZ-type cytokinin;
   (j) a gene encoding a protein that consists of the amino acid sequence shown in SEQ ID NO: 18, 20, 22, or 24;
   (k) a gene encoding a protein that consists of an amino acid sequence derived from the amino acid sequence shown in SEQ ID NO: 18, 20, 22 or 24 by deletion, substitution, or addition of 1 or several amino acids, and has activity to catalyze conversion from an iP-type cytokinin to a tZ-type cytokinin; and
   (l) a gene encoding a protein that consists of an amino acid sequence having 70% or higher identity with the amino acid sequence shown in SEQ ID NO: 18, 20, 22, or 24, and has activity to catalyze conversion from an iP-type cytokinin to a tZ-type cytokinin
[6] A method for increasing plant biomass, comprising overexpressing an active cytokinin synthase gene and a cytokinin hydroxylase gene in a plant body.
[7] The method of [6], wherein the active cytokinin synthase gene is a LOG gene.
[8] The method of [6], wherein the cytokinin hydroxylase gene is a CYP735A gene.
[9] The method of [6], wherein the active cytokinin synthase gene is any one of the following genes (a) to (f):
   (a) a gene comprising DNA that consists of the nucleotide sequence shown in SEQ ID NO: 1, 3, 5, 7, 9, 11, 13, or 15;
   (b) a gene comprising DNA that hybridizes under stringent conditions to DNA consisting of a nucleotide sequence complementary to DNA consisting of the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
   (c) a gene comprising DNA that consists of a nucleotide sequence having 70% or higher identity with the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
   (d) a gene encoding a protein that consists of the amino acid sequence shown in SEQ ID NO: 2, 4, 6, 8, 10, 12, 14, or 16;
   (e) a gene encoding a protein that consists of an amino acid sequence derived from the amino acid sequence shown in SEQ ID NO: 2, 4, 6, 8, 10, 12, 14 or 16 by deletion, substitution, or addition of 1 or several amino acids, and has activity to catalyze a reaction for synthesizing an active cytokinin from a nucleotide cytokinin; and
   (f) a gene encoding a protein that consists of an amino acid sequence having 70% or higher identity with the amino acid sequence shown in SEQ ID NO: 2, 4, 6, 8, 10, 12, 14, or 16, and has activity to catalyze a reaction for synthesizing an active cytokinin from a nucleotide cytokinin.
[10] The method of [6], wherein the cytokinin hydroxylase gene is any one of the following genes (g) to (l):
   (g) a gene comprising DNA that consists of the nucleotide sequence shown in SEQ ID NO: 17, 19, 21, or 23;
   (h) a gene comprising DNA that hybridizes under stringent conditions to DNA consisting of a nucleotide sequence complementary to DNA consisting of the nucleotide sequence shown in SEQ ID NO: 17, 19, 21 or 23, and encodes a protein having activity to catalyze conversion from an iP-type cytokinin to a tZ-type cytokinin;
   (i) a gene comprising DNA that consists of a nucleotide sequence having 70% or higher identity with the nucleotide sequence shown in SEQ ID NO: 17, 19, 21 or 23, and encodes a protein having activity to catalyze conversion from an iP-type cytokinin to a tZ-type cytokinin;
   (j) a gene encoding a protein that consists of the amino acid sequence shown in SEQ ID NO: 18, 20, 22, or 24;
   (k) a gene encoding a protein that consists of an amino acid sequence derived from the amino acid sequence shown in SEQ ID NO: 18, 20, 22 or 24 by deletion, substitution, or addition of 1 or several amino acids, and has activity to catalyze conversion from an iP-type cytokinin to a tZ-type cytokinin; and
   (l) a gene encoding a protein that consists of an amino acid sequence having 70% or higher identity with the amino acid sequence shown in SEQ ID NO: 18, 20, 22, or 24, and has activity to catalyze conversion from an iP-type cytokinin to a tZ-type cytokinin.
[11] A transgenic plant, wherein an active cytokinin synthase gene and a cytokinin hydroxylase gene are introduced to increase biomass.
[12] The transgenic plant of [11], wherein the active cytokinin synthase gene is a LOG gene.
[13] The transgenic plant of [11], wherein the cytokinin hydroxylase gene is a CYP735A gene.
[14] The transgenic plant of [11], wherein the active cytokinin synthase gene is any one of the following genes (a) to (f):
   (a) a gene comprising DNA that consists of the nucleotide sequence shown in SEQ ID NO: 1, 3, 5, 7, 9, 11, 13, or 15;
   (b) a gene comprising DNA that hybridizes under stringent conditions to DNA consisting of a nucleotide sequence complementary to DNA consisting of the nucleotide sequence shown in SEQ ID NO:, 1, 3, 5, 7, 9, 11, 13, or 15, and encodes a protein having activity to catalyze a reaction for synthesizing an active cytokinin from a nucleotide cytokinin;
   (c) a gene comprising DNA that consists of a nucleotide sequence having 70% or higher identity with the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
   (d) a gene encoding a protein that consists of the amino acid sequence shown in SEQ ID NO: 2, 4, 6, 8, 10, 12, 14 or 16;
   (e) a gene encoding a protein that consists of an amino acid sequence derived from the amino acid sequence shown in SEQ ID NO: 2, 4, 6, 8, 10, 12, 14 or 16 by deletion, substitution, or addition of 1 or several amino acids, and has activity to catalyze a reaction for synthesizing an active cytokinin from a nucleotide cytokinin; and
   (f) a gene encoding a protein that consists of an amino acid sequence having 70% or higher identity with the amino acid sequence shown in SEQ ID NO: 2, 4, 6, 8, 10, 12, 14, or 16, and has activity to catalyze a reaction for synthesizing an active cytokinin from a nucleotide cytokinin.
[15] The transgenic plant of [11], wherein the cytokinin hydroxylase gene is any one of the following genes (g) to (l):
   (g) a gene comprising DNA that consists of the nucleotide sequence shown in SEQ ID NO: 17, 19, 21, or 23;
   (h) a gene comprising DNA that hybridizes under stringent conditions to DNA consisting of a nucleotide sequence complementary to DNA consisting of the nucleotide sequence shown in SEQ ID NO: 17, 19, 21, or 23, and encodes a protein having activity to catalyze conversion from an iP-type cytokinin to a tZ-type cytokinin;
   (i) a gene comprising DNA that consists of a nucleotide sequence having 70% or higher identity with the nucleotide sequence shown in SEQ ID NO: 17, 19, 21, or 23, and encodes a protein having activity to catalyze conversion from an iP-type cytokinin to a tZ-type cytokinin;
   (j) a gene encoding a protein that consists of the amino acid sequence shown in SEQ ID NO: 18, 20, 22, or 24;
   (k) a gene encoding a protein that consists of an amino acid sequence derived from the amino acid sequence shown in SEQ ID NO: 18, 20, 22 or 24 by deletion, substitution, or addition of 1 or several amino acids, and has activity to catalyze conversion from an iP-type cytokinin to a tZ-type cytokinin; and
   (l) a gene encoding a protein that consists of an amino acid sequence having 70% or higher identity with the amino acid sequence shown in SEQ ID NO: 18, 20, 22, or 24, and has activity to catalyze conversion from an iP-type cytokinin to a tZ-type cytokinin.

### BRIEF DESCRIPTION OF DRAWINGS

FIG. 1 shows the amounts of cytokinins produced within transgenic plants overexpressing AtLOG and CYP735A genes (unit (pmol/g FW): the amount of cytokinin (pmol) per gram of fresh weight (FW) of a plant body; tZ: trans-zeatin, tZR: tZ riboside; tZRPs: tZR phosphates; tZ7G: tZ-7-N-glucoside; tZOG: tZ-O-glucoside; iP: N⁶-(Δ²-isopentenyl)adenine; iPR: iP riboside; iPRPs: iPR phosphates; iP7G: iP-7-N-glucoside; and iP9G: iP-9-N-glucoside).

FIG. 2 is a model diagram showing synthesis mechanisms of active cytokinins (iP-type and tZ-type).

FIG. 3 shows the morphology of the aerial parts of transgenic plants overexpressing AtLOG and CYP735A genes in week 4 after germination (scale: 1 cm).

FIG. 4A shows the morphology of transgenic plants overexpressing AtLOG and CYP735A genes in week 7 after germination. FIG. 4B shows the same on month 2 after germination (scale: 5 cm).

FIG. 5 shows the morphology of the inflorescences of transgenic plants overexpressing AtLOG and CYP735A genes in week 7 after germination (scale: 1 cm).

The present application claims the priority to Japanese Patent Application No. 2010-241241 filed on Oct. 27, 2010, and the contents of the patent application are herein incorporated by reference.

### BEST MODES FOR CARRYING OUT THE INVENTION

The method for increasing plant biomass of the present invention is characterized by overexpressing “an active cytokinin synthase gene” and “a cytokinin hydroxylase gene,” which are two genes involved in cytokinin synthesis and activation, in a plant body.

The above term “active cytokinin synthase gene” refers to an enzyme gene that catalyzes a reaction for synthesizing an active cytokinin from a nucleotide cytokinin. Preferably, such an active cytokinin synthase gene is an LOG gene.

Examples of the LOG gene include a rice LOG gene (Accession number: AK071695) and Arabidopsis thaliana LOG homolog genes having high homology with the rice LOG gene, such as At2g28305 (AtLOG1, Accession number: NM_{—}128389), At2g35990 (AtLOG2, Accession number: NM_{—}129158), At2g37210 (AtLOG3, Accession number: NM_{—}129277), At3g53450 (AtLOG4, Accession number: NM_{—}115205), At4g35190 (AtLOG5, Accession number: NM_{—}119685), At5g06300 (AtLOG7, Accession number: NM_{—}120713), and At5g11950 (AtLOG8, Accession number: NM_{—}203039). The nucleotide sequences of the rice LOG gene, and AtLOG1, 2, 3, 4, 5, 7, and 8 genes are shown in SEQ ID NOS: 1, 3, 5, 7, 9, 11, 13, and 15, respectively, and the genes encode the amino acid sequences shown in SEQ ID NOS: 2, 4, 6, 8, 10, 12, 14, and 16, respectively.

In the present invention, as a LOG gene, in addition to a gene consisting of the nucleotide sequence shown in SEQ ID NO: 1, 3, 5, 7, 9, 11, 13, or 15, a gene consisting of a nucleotide sequence analogous to the nucleotide sequence shown in SEQ ID NO: 1, 3, 5, 7, 9, 11, 13, or 15, can also be used herein, as long as it has functions equivalent to those of the relevant gene. Therefore, examples of a LOG gene used in the present invention include a homolog LOG gene that consists of a nucleotide sequence analogous to the nucleotide sequence of any one of SEQ ID NOS above, and encodes a protein having activity to catalyze a reaction for synthesizing an active cytokinin from a nucleotide cytokinin. Such a homolog LOG gene may be prepared from nature or artificially prepared. For example, such a homolog LOG gene may be a homolog (including an ortholog and a paralog) of the nucleotide sequence of any one of the above sequence identification numbers or may have a mutation artificially introduced therein.

Here, the expression “activity to catalyze a reaction for synthesizing an active cytokinin from a nucleotide cytokinin” refers to activity to catalyze a reaction to dissociate ribose 5′-monophosphate from a nucleotide cytokinin so as to synthesize an active cytokinin in a base form.

In the present invention, examples of the “nucleotide cytokinin” include isopentenyladenine riboside 5′-monophosphate (iPRMP), trans-zeatin riboside 5′-monophosphate (tZRMP), dihydrozeatin riboside 5′-monophosphate (DZRMP), and cis-zeatin riboside 5′-monophosphate (cZRMP). Examples of the “active cytokinin” include isopentenyladenine (N⁶-(Δ²-isopentenyl)adenine (iP)), trans-zeatin (tZ), dihydrozeatin (DZ), and cis-zeatin (cZ), which are prepared by dissociation of ribose and dissociation of 5′-monophosphate of the above nucleotide cytokinin.

Specific examples thereof include the following homolog LOG genes.
(i) A gene comprising DNA that hybridizes under stringent conditions to DNA consisting of a nucleotide sequence complementary to DNA consisting of the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin.
(ii) A gene comprising DNA that consists of a nucleotide sequence having 70% or higher identity with the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin.
(iii) A gene encoding a protein that consists of an amino acid sequence derived from the amino acid sequence shown in SEQ ID NO: 2, 4, 6, 8, 10, 12, 14 or 16 by deletion, substitution, or addition of 1 or several amino acids, and has activity to catalyze a reaction for synthesizing an active cytokinin from a nucleotide cytokinin.
(iv) A gene encoding a protein that consists of an amino acid sequence having 70% or higher identity with the amino acid sequence shown in SEQ ID NO: 2, 4, 6, 8, 10, 12, 14, or 16, and has activity to catalyze a reaction for synthesizing an active cytokinin from a nucleotide cytokinin.

The term “stringent conditions” as used herein refers to the conditions under which so-called specific hybrids are formed but non-specific hybrids are not formed. A person skilled in the art can adequately select the stringent hybridization conditions by referring to Sambrook et al., Molecular Cloning, A Laboratory Manual, 3rd Ed., Cold Spring Harbor Laboratory, 2001. For example, hybridization is carried out by performing pre-hybridization in a hybridization solution containing 25% formamide, or 50% formamide for more stringent conditions, 4×SSC, 50 mM HEPES (pH 7.0), 10×Denhart's solution, and 20 μg/ml denatured salmon sperm DNA at 42° C. overnight, adding a labeled probe thereto, and incubating the resultant at 42° C. overnight. In the subsequent step of washing, the washing solution and temperature conditions are approximately “1×SSC, 0.1% SDS, 37° C.,” approximately “0.5×SSC, 0.1% SDS, 42° C.” for more stringent conditions, and approximately “0.2×SSC, 0.1% SDS, 65° C.” for even more stringent conditions. The degree of stringency is increased as the temperature becomes higher and the salt concentration becomes lower. This enables isolation of a gene with higher identity.

It should be noted that the combinations of SSC, SDS and temperature conditions described above are examples. A person skilled in the art can adequately combine the above or other factors that determine the hybridization stringency (e.g., probe concentration, probe length, and hybridization duration) to realize stringency similar to that described above.

DNA obtained by hybridization carried out under the above stringent conditions usually has high identity to DNA represented by the nucleotide sequence shown in SEQ ID NO: 1, 3, 5, 7, 9, 11, 13 or 15. The term “high identity” as used herein refers to the sequence identity of 70% or higher, preferably 80% or higher, 85% or higher, more preferably 90% or higher, further preferably 95% or higher, and most preferably 97% or higher (e.g., 98% to 99%) to any of the nucleotide sequences shown in the above sequence identification numbers.

In relation to the expression “amino acid sequence derived by deletion, substitution, or addition of 1 or several amino acids”, the number of amino acids that may be deleted, substituted or added is the number of amino acids that can be deleted, substituted or added in accordance with a known method for preparing mutant proteins, such as site-directed mutagenesis. The number is not limited as long as the aforementioned activity is maintained. Usually, the number is for example 1 to 20, preferably 1 to 10, and more preferably 1 to 5. The term “mutation” as used herein primarily means a mutation that is artificially introduced in accordance with a known method for preparing mutant proteins, although a naturally occurring similar mutation may be employed.

The term “70% or higher identity” used in relation to the amino acid sequence refers to sequence identity of preferably 80% or higher, 85% or higher, more preferably 90% or higher, further preferably 95% or higher, and most preferably 97% or higher (e.g., 98% to 99%). Identity of sequence (amino acid sequence, nucleotide sequence) can be determined using the FASTA search or the BLAST search.

The LOG gene used in the present invention can be prepared using a known technique. For example, total mRNA may be prepared from an Arabidopsis thaliana tissue extract, primers may be designed based on the nucleotide sequence shown in the above sequence identification number, and full-length cDNA of the nucleotide sequence shown in the above sequence identification number can be obtained by performing the RACE method or the like. Alternatively, a cDNA library may be prepared from an Arabidopsis thaliana tissue extract, a probe may be designed based on the nucleotide sequence shown in the above sequence identification number, and the LOG gene of interest can be obtained using the hybridization method. Further, the LOG gene may be artificially synthesized based on the nucleotide sequence shown in the above sequence identification number.

A person skilled in the art can readily obtain a homolog of the LOG gene by referring to, for example, Molecular Cloning (Sambrook, J. et al., Molecular Cloning: A Laboratory Manual 3rd ed., Cold Spring Harbor Laboratory Press, 10 Skyline Drive Plainview, N.Y., 2001).

For example, deletion, addition and substitution of an amino acid can be carried out by introducing a mutation into a gene encoding the above protein using a technique known in the art. Mutation can be introduced into a gene by a known technique such as the Kunkel method or the Gapped duplex method, or a method in accordance therewith. For example, a kit for introducing mutation that utilizes the site-directed mutagenesis method (e.g., Mutant-K (TAKARA) or Mutant-G (TAKARA)), or the kit of LA PCR in vitro Mutagenesis series (TAKARA)) can be used. Alternatively, a sequence having a mutation being introduced into the nucleotide sequence shown in the above sequence identification number may be synthesized using a commercially available nucleic acid synthesis apparatus.

On the other hand, the term “cytokinin hydroxylase gene” to be co-expressed with the “active cytokinin synthase gene” refers to an enzyme gene that catalyzes conversion from an iP-type cytokinin to a tZ-type cytokinin. Preferably, the cytokinin hydroxylase gene is a CYP735A gene.

Examples of the CYP735A gene include an Arabidopsis thaliana CYP735A1 gene, an Arabidopsis thaliana CYP735A2 gene, a rice CYP735A3 gene, and a rice CYP735A4 gene. The CYP735A1 gene, the CYP735A2 gene, the CYP735A3 gene, and the CYP735A4 gene have the nucleotide sequences shown in SEQ ID NOS: 17, 19, 21 and 23, respectively, and encode the amino acid sequences shown in SEQ ID NOS: 18, 20, 22 and 24, respectively. The CYP735A1 gene and the CYP735A2 gene are known genes isolated from Arabidopsis thaliana, and the nucleotide sequences thereof are registered in the DNA Data Bank of Japan (DDBJ) under accession nos. BX832759 and BT011622, respectively. The AGI codes thereof are disclosed as At5g38450 and At1g67110, respectively (Arabidopsis CYP735A1 and CYP735A2 encode cytokinin hydroxylases that catalyze the biosynthesis of trans-zeatin. J. Biol. Chem. 279: 41866-41872, July 2004, Takei, K., Yamaya, T. and Sakakibara, H.). Furthermore, the amino acid sequences encoded by the CYP735A1 gene and the CYP735A2 gene are registered in the GenBank under accession nos. NP_{—}198661 and NP_{—}176882, respectively. Moreover, the CYP735A3 gene and the CYP735A4 gene are known genes isolated from rice (David R. David R. Nelson, et al., (2004). Comparative Genomics of Rice and Arabidopsis. Analysis of 727 Cytochrome P450 Genes and Pseudogenes from a Monocot and a Dicot, Plant Physiology, Vol. 135, pp. 756-772).

As the CYP735A gene, similarly, in addition to a gene consisting of the nucleotide sequence shown in SEQ ID NO: 17, 19, 21 or 23, a gene consisting of a nucleotide sequence analogous to the nucleotide sequence shown in SEQ ID NO: 17, 19, 21 or 23 can be used, as long as it has functions equivalent to those of the relevant gene. Therefore, examples of the CYP735A gene used in the present invention include a homolog CYP735A gene consisting of a nucleotide sequence analogous to the nucleotide sequence of any one of the above sequence identification numbers and encoding a protein that has activity to catalyze conversion from an iP-type cytokinin to a tZ-type cytokinin.

Specific examples thereof include the following homolog CYP735A genes:
(i) a gene comprising DNA that hybridizes under stringent conditions to DNA consisting of a nucleotide sequence complementary to DNA consisting of the nucleotide sequence shown in SEQ ID NO: 17, 19, 21, or 23, and encodes a protein having activity to catalyze conversion from an iP-type cytokinin to a tZ-type cytokinin;
(ii) a gene comprising DNA that consists of a nucleotide sequence having 70% or higher identity with the nucleotide sequence shown in SEQ ID NO: 17, 19, 21, or 23, and encodes a protein having activity to catalyze conversion from an iP-type cytokinin to a tZ-type cytokinin;
(iii) a gene encoding a protein that consists of an amino acid sequence derived from the amino acid sequence shown in SEQ ID NO: 18, 20, 22 or 24 by deletion, substitution, or addition of 1 or several amino acids, and has activity to catalyze conversion from an iP-type cytokinin to a tZ-type cytokinin; and
(iv) a gene encoding a protein that consists of an amino acid sequence having 70% or higher identity with the amino acid sequence shown in SEQ ID NO: 18, 20, 22, or 24, and has activity to catalyze conversion from an iP-type cytokinin to a tZ-type cytokinin.

Furthermore, the meaning of “stringent” or “sequence identity” used for the above CYP735A gene is as defined for the above LOG gene.

Here, the expression “activity to catalyze conversion from an iP-type cytokinin to a tZ-type cytokinin” refers to activity to catalyze a reaction for hydroxylation of an iP (isopentenyladenine (iP))-type cytokinin so as to convert it to a tZ (trans-zeatin)-type cytokinin. In the present invention, examples of the “iP-type cytokinin” include isopentenyladenine riboside 5′-monophosphate (iPRMP), isopentenyladenine riboside 5′-diphosphate (iPRDP), and isopentenyladenine riboside 5′-triphosphate (iPRTP). Examples of the “tZ-type cytokinin” include trans-zeatin riboside 5′-monophosphate (tZRMP), trans-zeatin riboside 5′-diphosphate (tZRDP), and trans-zeatin riboside 5′-triphosphate (tZRTP), which are prepared by adding a hydroxy group to each of the above iP-type cytokinins. These iP-type cytokinins and tZ-type cytokinins are referred to as “iP-type cytokinin nucleotides” and “tZ-type cytokinin nucleotides,” respectively. Although the CYP735A gene is not directly involved in its synthesis, in the present specification, examples of the “iP-type cytokinin” include isopentenylriboside (iPR) and isopentenyladenine (iP), which are prepared by dephosphorylation and dissociation of ribose of the above iP-type cytokinin nucleotide. Examples of the “tZ-type cytokinin” include trans-zeatin riboside (tZR) and trans-zeatin (tZ), which are prepared by dephosphorylation and dissociation of ribose of the above tZ-type cytokinin nucleotide. Furthermore, examples of “iP-type cytokinin” and “tZ-type cytokinin” include glycosides. Examples of glycosides of the iP-type cytokinins include isopentenyladenine-7-glucoside (iP7G) and isopentenyladenine-9-glucoside (iP9G). Examples of glycosides of the tZ-type cytokinins include trans-zeatin-7-glucoside (tZ7G), trans-zeatin-9-glucoside (tZ9G), trans-zeatin-O-glucoside (tZOG), and trans-zeatinriboside-O-glucoside (tZROG).

A transgenic plant overexpressing (excessive co-expression) the above 2 genes, “active cytokinin synthase gene” and “cytokinin hydroxylase gene” can be prepared by introducing the 2 genes into plant cells so that co-expression is possible, and then regenerating plant bodies from the plant cells. For example, a recombinant vector is constructed by ligating (inserting) a sequence required for co-expression of the 2 genes and then the vector is introduced into plant cells. The term “co-expression” means that genes encoding two different proteins are operably linked to the same or different promoters and then the genes are expressed simultaneously. Therefore, as long as co-expression is possible, the above genes may be integrated into the same vector or different vectors, and then they are used as binary vectors. When genes are integrated into the same vector, a sequence for ribosomal protein rebinding (IRES: Internal Ribosome Entry Site) may be inserted into a site between the genes encoding two different proteins. IRES may be derived from a virus or a plant to be transformed. As IRES derived from a plant to be transformed, a predetermined sequence of 18SrDNA can be used (JP Patent Publication (kokai) No. 2005-198625 A), but the examples thereof are not limited thereto. Alternatively, a transgenic plant can also be prepared by crossing a transformant overexpressing a 1^{st} gene, which is obtained by introducing only one (the 1^{st} gene) of 2 genes into plant cells, with a transformant overexpressing a 2^{nd} gene, which is obtained by introducing the other gene (the 2^{nd} gene) alone into plant cells.

The recombinant vector used for plant transformation can be constructed by introducing the above two genes (hereinafter referred to as “target genes”) into an adequate vector. Examples of vectors that can be preferably used include pBI, pPZP, pSMA, and pCAMBIA vectors which can introduce the target gene into a plant via Agrobacterium. Use of pBI binary vectors or intermediate vectors is particularly preferable, and examples thereof include pBI121, pBI101, pBI101.2, and pBI101.3. The term “binary vector” refers to a shuttle vector replicable in Escherichia coli and Agrobacterium. When a plant is infected with Agrobacterium that harbors a binary vector, DNA located in a region defined by the border sequences (LB sequence and RB sequence) on the vector can be integrated into plant nuclear DNA. On the other hand, pUC vectors are capable of directly introducing a gene into a plant, and examples thereof include pUC18, pUC19 and pUC9. Further, plant virus vectors, such as cauliflower mosaic virus (CaMV), bean golden mosaic virus (BGMV), and tobacco mosaic virus (TMV) vectors, can also be used.

When a binary vector plasmid is used, a target gene is inserted into a site between border sequences (LB and RB sequences) of the binary vector, and the recombinant vector is amplified in Escherichia coli. Subsequently, the amplified recombinant vector is introduced into, for example, Agrobacterium tumefaciens GV3101, C58, LBA4404, EHA101 or EHA105, or Agrobacterium rhizogenes LBA1334, by the electroporation method or the like, and the Agrobacterium is used for plant transduction.

For inserting a target gene into a vector, one may employ a method in which purified DNA is first cleaved with an adequate restriction enzyme and the cleaved fragment is then inserted into a restriction enzyme site or multicloning site of adequate vector DNA to connect the fragment to the vector.

In addition, it is necessary that a target gene be integrated into a vector in such a manner that the gene is able to exert its functions. Thus, a promoter, an enhancer, a terminator, a replication origin that allows the use of a binary vector (e.g., a replication origin derived from Ti or Ri plasmid), a selection marker gene, and the like can be connected to a site upstream, inside, or downstream of the target gene in the vector.

A “promoter” may not be derived from a plant, provided that it is DNA that can function in a plant cell and bring about expression in a given tissue or at a given growth stage of a plant. Specific examples include cauliflower mosaic virus (CaMV) 35S promoter, nopaline synthase gene promoter (Pnos), maize-derived ubiquitin promoter, rice-derived actin promoter, and tobacco-derived PR protein promoter. When a target gene is to be expressed specifically in a given organ, a promoter that is expressed in a tissue-specific manner can be used. For example, the biomass-increasing effect of the target gene of the present invention can be efficiently attained by using a flowering stem-specific gene promoter.

An enhancer is used, for example, for increasing the expression efficiency of a target gene. Examples thereof include an enhancer region that comprises an upstream sequence in the CaMV 35S promoter.

Any sequence may be used as a terminator, provided that it can terminate transcription of a gene transcribed by a promoter. Examples thereof include terminators of the nopaline synthase (NOS) gene, the octopine synthase (OCS) gene, and the CaMV 35S RNA gene.

Examples of selection marker genes include ampicillin-resistant gene, neomycin-resistant gene, hygromycin-resistant gene, bialaphos-resistant gene, and dihydrofolate reductase gene.

The selection marker gene may be connected to a single plasmid together with a target gene as described above to prepare a recombinant vector. Alternatively, a recombinant vector obtained by connecting the selection marker gene to a plasmid and a recombinant vector obtained by connecting a target gene may be separately prepared. When the vectors are separately prepared, the vectors are co-transfected (co-introduced) into a host.

The transgenic plant of the present invention can be produced by introducing the above gene or recombinant vector (hereinafter, collectively referred to as “target gene”) into a target plant. In the present invention, the term “introduction of a gene” means that a target gene is introduced into cells of a host plant in a manner that allows the gene to express using, for example, a known genetic engineering technique. The introduced gene may be integrated into the genomic DNA of a host plant or may be present being comprised in a foreign vector.

As a method for introducing the target gene into a plant as described above, one of a variety of methods that have been reported and established can be adequately utilized. Examples thereof include the Agrobacterium method, the PEG-calcium phosphate method, the electroporation method, the liposome method, the particle gun method, and the microinjection method. When the Agrobacterium method is employed, a protoplast, a tissue section, or a plant body as it is (i.e., the in planta method) may be used. When a protoplast is used, the introduction can be carried out using a method in which the protoplast is co-cultured with Agrobacterium harboring a Ti plasmid or an Ri plasmid (for Agrobacterium tumefaciens or Agrobacterium rhizogenes, respectively), or the protoplast is fused to Agrobacterium which has been converted to a spheroplast (the spheroplast method). When a tissue section is used, the introduction can be carried out using a method in which an aseptically cultured leaf disc of a target plant or a callus (cultured undifferentiated cell) is infected. When the in planta method using a seed or a plant body is employed (i.e., in a system that does not involve tissue culture with the addition of plant hormones), the introduction can be carried out by direct treatment of an imbibed seed, a young seedling, a potted plant, or the like with Agrobacterium. These plant transformation methods can be carried out in accordance with the descriptions of general textbooks such as “Shinban, Model shokubutsu no jikken protocol, Idengakuteki shuhou kara genome kaiseki made (New edition, Experimental protocols for model plants, From genetic engineering technique to genome analysis), 2001, supervised by Isao Shimamoto & Kiyotaka Okada, Shujunsha.”

One can confirm whether or not a target gene has been incorporated into a plant using the PCR method, the Southern hybridization method, the Northern hybridization method, the Western blotting method or the like. For example, DNA is prepared from a transgenic plant, primers specific for the target gene are designed, and PCR is then carried out. After PCR has been carried out, the amplification product is subjected to agarose gel electrophoresis, polyacrylamide gel electrophoresis, capillary electrophoresis or the like, and stained with ethidium bromide, a SYBR Green solution or the like. Transformation can be confirmed based on detection of the amplification product as a single band. Alternatively, the amplification product can be detected by carrying out PCR with the use of primers that have been labeled with a fluorescent dye or the like beforehand. Further, one may use a method in which the amplification product is bound to a solid phase such as a microplate, and confirmed using fluorescence, an enzymatic reaction or the like. Further, one may confirm that a target gene introduced into a plant cell is expressed (that is, the plant is transformed) by extracting proteins from the plant cell, fractionating the proteins by two-dimensional electrophoresis, and detecting a band of the protein encoded by the target gene.

Alternatively, a vector in which one of a variety of reporter genes (e.g., a gene for β-glucuronidase (GUS), luciferase (LUC), green fluorescent protein (GFP), chloramphenicol acetyltransferase (CAT) or β-galactosidase (LacZ)) is connected downstream of a target gene is prepared. Agrobacterium into which the aforementioned vector has been introduced is used to transform a plant in a manner similar to that as described above. Then, the expression of the reporter gene is measured. Thereby, transformation of the plant can be confirmed.

The plant used for transformation in the present invention may be a monocotyledonous plant or a dicotyledonous plant. Examples of plants used for transformation in the present invention include plants belonging to the family Brassicaceae, the family Gramineae, the family Solanaceae, the family Leguminosae, the family Compositae, the family Arecaceae, the family Anacardiaceae, the family Cucurbitaceae, the family Rosaceae, the family Caryophyllaceae, the family Salicaceae, the family Myrtaceae, and the family Liliaceae, for example (see below).
The family Brassicaceae: Arabidopsis thaliana (Arabidopsis thaliana), rapeseed (Aburana) (Brassica rapa, Brassica napus), cabbage (Brassica oleracea var. capitata), rapeseed (Natane) (Brassica rapa, Brassica napus), rape blossoms (Brassica rapa, Brassica napus), Chinese cabbage (Brassica rapa var. pekinensis), qing-geng-cai (Brassica rapa var. chinensis), turnip (Brassica rapa var. rapa), nozawana (Brassica rapa var. hakabura), potherb mustard (Brassica rapa var. lancinifolia), komatsuna (Brassica rapa var. peruviridis), Chinese cabbage (Paku Choi) (Brassica rapa var. chinensis), radish (Brassica Raphanus sativus), wasabi (Wasabia japonica), etc.
The family Gramineae: corn (Zea mays), rice (Oryza sativa), barley (Hordeum vulgare), wheat (Triticum aestivum), bamboo (Phyllostachys), sugarcane (Saccharum officinarum), napier grass (Pennisetum purpureum), Erianthus (Erianthus ravennae), Japanese silver grass (Miscanthus virgatum), Sorghum (Sorghum), switchgrass (Panicum), etc.
The family Solanaceae: tobacco (Nicotiana tabacum), eggplant (Solanum melongena), potato (Solaneum tuberosum), tomato (Lycopersicon lycopersicum), pepper (Capsicum annuum), petunia (Petunia), etc.
The family Leguminosae: soybean (Glycine max), pea (Pisum sativum), fava bean (Vicia faba), Japanese wisteria (Wisteria floribunda), peanut (Arachis. hypogaea), Lotus japonicus (Lotus corniculatus var. japonicus), common bean (Phaseolus vulgaris), azuki (Vigna angularis), acacia (Acacia), etc.
The family Compositae: Chrysanthemum (Chrysanthemum morifolium), sunflower (Helianthus annuus), etc.
The family Arecaceae: oil palm (Elaeis guineensis, Elaeis oleifera), coconut (Cocos nucifera), Date Palm (Phoenix dactylifera), wax palm (Copernicia)
The family Anacardiaceae: hazenoki (Rhus succedanea), cashew (Anacardium occidentale), poison oak (Toxicodendron vernicifluum), mango (Mangifera indica), pistachio (Pistacia vera), etc.
The family Cucurbitaceae: pumpkin (Cucurbita maxima, Cucurbita moschata, Cucurbita pepo), cucumber (Cucumis sativus), Trichosanthes (karasu uri) (Trichosanthes cucumeroides), gourd (Lagenaria siceraria var. gourda), etc.
The family Rosaceae: almond (Amygdalus communis), rose (Rosa), strawberry (Fragaria), cherry (Prunus), apple (Malus pumila var. domestica), etc.
The family Caryophyllaceae: carnation (Dianthus caryophyllus), etc.
The family Salicaceae: poplar (Populus trichocarpa, Populus nigra, Populus tremula), etc.
The family Myrtaceae: eucalyptus (Eucalyptus camaldulensis, Eucalyptus grandis), etc.
The family Liliaceae: tulip (Tulipa), lily (Lilium), etc.

Examples of plant materials to be subjected to transformation in the present invention include: plant organs such as stems, leaves, seeds, embryos, ovules, ovaries and shoot apices; plant tissues such as anthers and pollens, and the sections thereof; undifferentiated calluses; and cultured plant cells such as protoplasts which are prepared by removing cell walls from the above by enzyme treatment. When the in planta method is employed, an imbibed seed or a whole plant body can be utilized.

According to the present invention, the term “transgenic plant” means any one of a whole plant body, a plant organ (e.g., leaf, petal, stem, root, grain or seed), a plant tissue (e.g., epidermis, phloem, parenchyma, xylem, or vascular bundle), or a cultured plant cell (e.g., callus).

When a cultured plant cell is to be used, an organ or an individual may be regenerated according to a known tissue culture method in order to regenerate a transformant from a resulting transformed cell. A person skilled in the art can readily carry out such a procedure using a method that is commonly known as a method of regenerating a plant body from a plant cell. For example, a plant body can be regenerated from a plant cell in the following manner.

At the outset, when a plant tissue or a protoplast is used as a plant material to be subjected to transformation, it is cultured in a medium for callus formation that has been sterilized after adding, for example, inorganic elements, vitamins, carbon sources, saccharides as energy sources or plant growth regulators (plant hormones, such as auxin, cytokinin, gibberellin, abscisic acid, ethylene, or brassinosteroid) to form a dedifferentiated callus which proliferates in an unstructured manner (hereinafter, this process is referred to as “callus induction”). The thus formed callus is transferred to a fresh medium containing plant growth regulators such as auxin, and then further proliferated (or subcultured).

Callus induction is carried out on a solid medium such as agar, and subculture is carried out, for example, in a liquid medium. Thereby, the cultivation can be carried out efficiently and in large quantities in the respective cases. Subsequently, the callus proliferated by the aforementioned subculture is cultured under adequate conditions to induce redifferentiation of an organ (hereinafter referred to as “induction of redifferentiation”), and a complete plant body is regenerated in the end. The induction of redifferentiation can be carried out by adequately setting the types and quantities of respective ingredients such as plant growth regulators (e.g., auxin) and carbon sources in the medium, light, temperature and the like. Such induction of redifferentiation results in formation of adventitious embryo, adventitious root, adventitious bud, adventitious shoot and the like, which further leads to growth into a complete plant body. Alternatively, storage may be conducted in a state prior to the formation of a complete plant body (e.g., encapsulated artificial seed, dry embryo, or freeze-dried cell or tissue).

The transgenic plants of the present invention also include plant bodies of progenies obtained by sexual or asexual reproduction of plant bodies having a gene of interest being introduced (including plant bodies regenerated from transformed cells or calluses), and portions of tissues or organs of the progeny plants (seeds, protoplasts, and the like). The transgenic plant of the present invention can be produced in large quantities by obtaining a reproductive material such as a seed or a protoplast, from a plant body transformed by introduction of the target gene, and then cultivating or culturing the same.

The transgenic plant obtained as described above exhibits increased biomass per plant as a result of excessive co-expression of the above 2 genes. In the present invention, the term “biomass” refers to the amount of a plant body or a part thereof existing within an arbitrary space at a given time. The term is used to encompass substances, foods, materials, fuels, resources and the like derived from said plant or parts thereof. Specifically, increased biomass refers to hypertrophy of a subterranean stem (rhizom, corm, tuber, bulb), a terrestrial stem, a flowering stem or a vine, hypertrophy of a seed, acceleration of elongation of stem length, plant length, culm length or ear length, or enlargement of a source organ such as a leaf. Biomass increased by the present invention is characterized in that the height of a plant body increases 1.2 fold or more, preferably 1.5 fold or more, more preferably 2 fold or more over a control wild-type plant. Alternatively, biomass increased by the present invention is characterized in that the number of blooming flowers increases 1.5 fold or more, preferably 2 fold or more, more preferably 3 fold or more, for example, over the same of a target wild-type plant.

The present invention is hereafter described in greater detail with reference to the following examples, although the present invention is not limited thereto.

### EXAMPLE 1

### Preparation of Transgenic Plants Overexpressing AtLOG and CYP735A Genes

(1) Preparation of transgenic plants overexpressing Arabidopsis thaliana LOG gene (AtLOGs) and CYP735A gene

The isolated cDNA of AtLOG4 was inserted to a site downstream of a tobacco mosaic virus 35S promoter of a plasmid pBI121 (Clontech) from which a GUS gene had been removed. The thus synthesized plasmid was introduced into Agrobacterium (Agrobacterium tumefaciens). Wild-type Arabidopsis thaliana was infected with Agrobacterium, for which the introduction of the plasmid had been confirmed by PCR. Collected seeds were sown on an MS medium containing kanamycin (50 ng/ml). Individual plants exhibiting resistance to kanamycin were selected by using the presence of a kanamycin-resistant gene (NPTII) in the T-DNA region of pBI121. A kanamycin-resistant line (hereinafter referred to as “35S::AtLOG4”) into which the genes had been introduced was selected. The mRNA of the 35S::AtLOG4 transgenic plant of the T1 generation extracted from rosette leaves and cDNA was synthesized by a reverse transcription reaction. Semi-quantitative RT-PCR analysis was carried out using the cDNA synthesized above as a template and primers for amplification of the AtLOG4 gene and the Actin2 gene. As a control, cDNA (WT) derived from wild-type rosette leaves was used. RT-PCR was carried out for 25 cycles for AtLOG4. As a result, a line confirmed to overexpress AtLOG4 was obtained.

A plant overexpressing each CYP735A gene was prepared in a manner similar to the above except that the cDNA of CYP735A1 and CYP735A2 genes was used instead of the cDNA of AtLOG4.
(2) Preparation of transgenic plants overexpressing two genes (AtLOG gene and CYP735A gene)

A crossing experiment was conducted in which the transgenic plant prepared in (1) overexpressing the AtLOG4 gene alone was crossed with the transgenic plant overexpressing both AtLOG4 and CYP735A genes for phenotype comparison. No phenotype change was observed in the case of the overexpression of the CYP735A gene alone, and thus this case was omitted from the experiment. With the use of the transgenic plant (35S::AtLOG4) overexpressing the AtLOG4 gene as a paternal plant, the pollens were adhered to each stigma of a wild-type plant from which flower organs other than pistils had been removed, and the plant overexpressing CYP735A1 and CYP735A2 genes (35S::CYP735A1 and 35S::CYP735A2). The subsequent development of “pods” was observed and thus successful artificial crossing was confirmed. F1 seeds (F1 wild type×35S::AtLOG4, F1 35S::CYP735A1×35S::AtLOG4, and F1 35S::CYP735A2×35S::AtLOG4) that had been developed after fructification were collected. Wild-type seeds and each F1 seed were sown on rock wool and then used for measuring the amounts of cytokinins produced within plants and observing phenotypes, as described later.

### EXAMPLE 2

### Amounts of Cytokinins in Transgenic Plants Overexpressing AtLOG and CYP735A Genes

The effects of the overexpression of the AtLOG and CYP735A genes on cytokinin metabolism were examined. Specifically, seeds of the wild-type, 35S::CYP735A1, 35S::CYP735A2, F1 wild type×35S::AtLOG4, F1 35S::CYP735A1×35S::AtLOG4, and F1 35S::CYP735A2×35S::AtLOG4 plants were sown on rock wool. On day 15 after germination, about 100 mg each of the aerial parts was collected and then the amounts of the iP-type cytokinin and the tZ-type cytokinin produced in each plant body sample were determined by high-performance liquid chromatography/tandem mass spectrometry (Waters; AQUITY UPLC System/Quattro Ultima Pt).

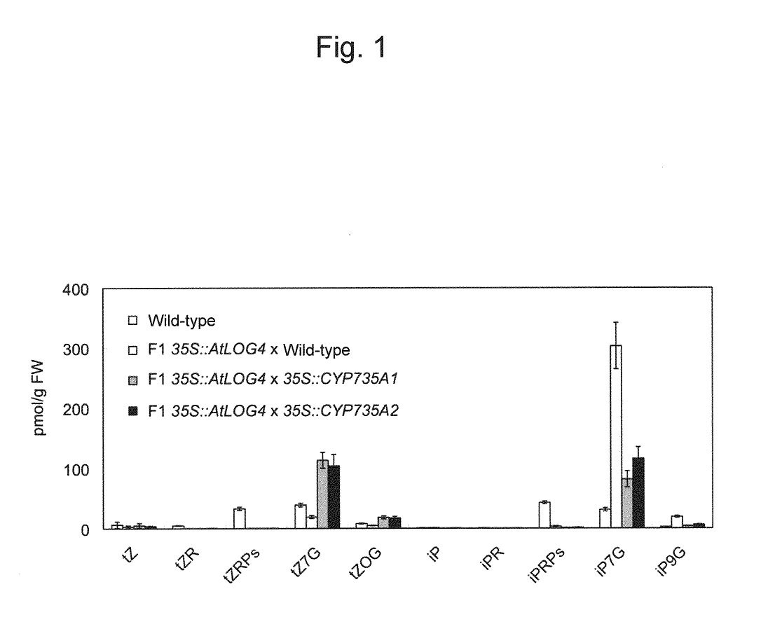

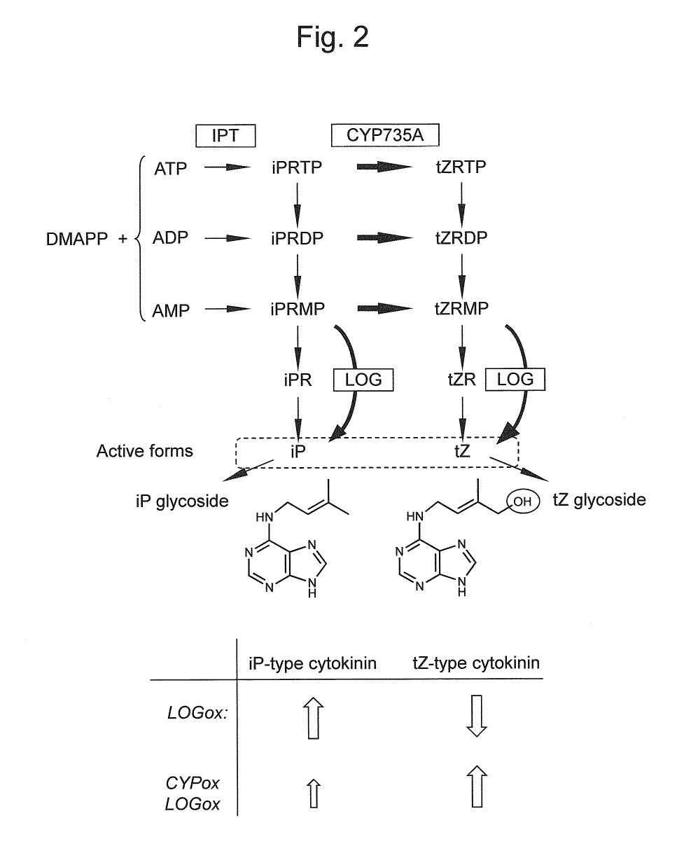

The results are shown in FIG. 1. In the case of the F1 35S::AtLOG4×wild type plant overexpressing AtLOG4 alone, no significant change was observed in the amounts of active cytokinins (iP and tZ) compared with wild-type plants, but significant decreases were observed in the amounts of iPRPs and tZRPs containing the substrate of the AtLOG gene product. It is known that excessively synthesized active cytokinins are inactivated by degradation or conversion to glycoside. In reference to this fact, in the case of the F1 35S::AtLOG4×wild-type plant, significant increases were observed in the amounts of iP7G and iP9G (glycosides of the iP-type cytokinin). On the other hand, in the case of the F1 35S::AtLOG4×wild-type plant, the amounts of tZ7G and tZOG (glycosides of the tZ-type cytokinin) had decreased to about a half of those of wild-type plants (see FIG. 1). The result was consistent with the result reported by Kuroha, T., Tokunaga, H., Kojima, M., Ueda, N., Ishida, T., Nagawa, S., Fukuda, H., Sugimoto, K., and Sakakibara, H. (2009). As a reason for the decreased amounts of glycosides of the tZ-type cytokinin in the plant overexpressing AtLOG4, the following mechanism is conceivable. The synthesis of the tZ-type cytokinin requires iPRMP (one of iPRPs), which is a substrate of the AtLOG gene product. However, in the case of a plant overexpressing AtLOG4, most of iPRMPs are used as substrates in an activation reaction for iP synthesis. Consequently, the synthesis amount of the tZ-type cytokinin decreased (see FIG. 2).

In contrast, in the cases of the F1 35S::CYP735A1×35S::AtLOG4 and F1 35S::CYP735A2×35S::AtLOG4, which were transgenic plants overexpressing both AtLOG and CYP735A genes, increases in the amounts of glycoside cytokinins (iP7G, iP9G, tZ7G and tZOG) were observed in both iP-type and tZ-type, and the rate of increase was always found to be about twice that of wild-type plants (see FIG. 1). As a reason for this result, the following mechanism is conceivable. Due to overexpression of both AtLOG and CYP735A genes, both the reaction in which iP is synthesized from PRMP using AtLOG as a catalyst and the reaction in which tZ-type cytokinin (tZRMP) is synthesized from iPRMP using CYP735A as a catalyst proceed, and thus, the activation reactions for both iP-type and tZ-type cytokinins were accelerated (see FIG. 2).

### EXAMPLE 3

### Phenotypes of Transgenic Plant Overexpressing AtLOG and CYP735A Genes

In order to examine the phenotypes when the AtLOG and CYP735A genes were overexpressed, the seeds of the wild-type, 35S::CYP735A1, 35S::CYP735A2, F1 wild-type×35S::AtLOG4, F1 35S::CYP735A1×35S::AtLOG4, and F1 35S::CYP735A2×35S::AtLOG4 plants were sown on rock wool.

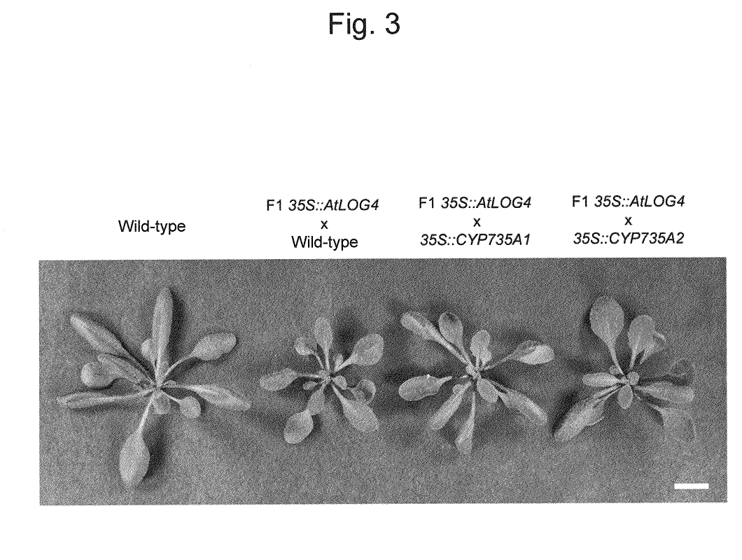

The phenotype of the aerial part of each plant body in week 4 after germination is shown in FIG. 3. Dwarfed rosette leaves were observed in the case of overexpression of AtLOG4 alone. Rosette leaves of a plant body overexpressing both AtLOG4 and CYP735A1 or CYP735A2 genes had almost the same size as that in the case of wild-type plants.

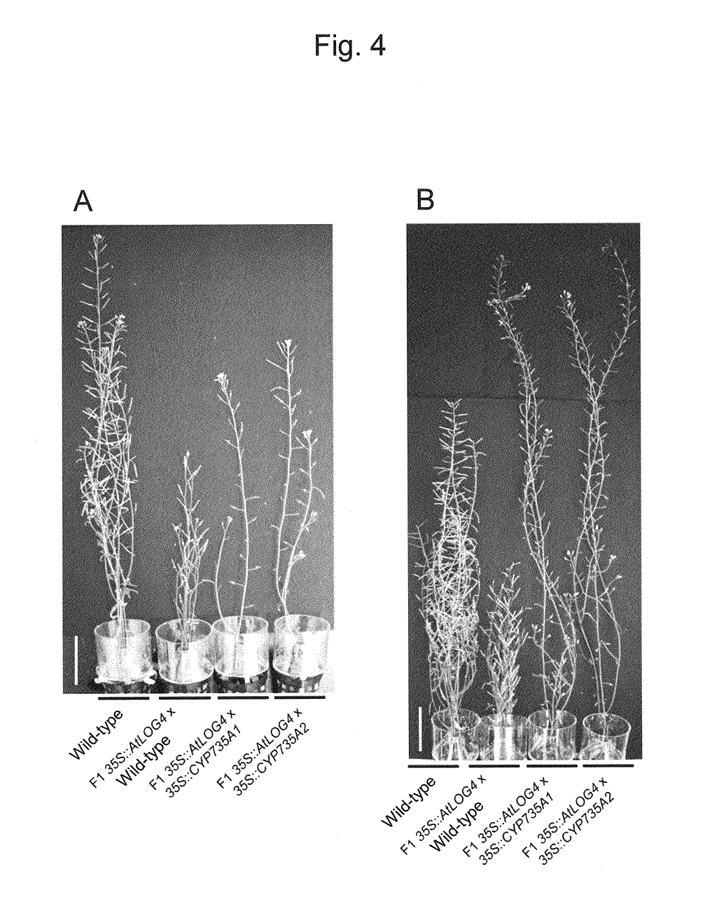

The phenotype of the aerial part of each plant body in week 7 after germination (A) and the same on month 2 after germination (B) are shown in FIG. 4. The height of the plant overexpressing AtLOG4 alone after bolting was always found to be about a half of that of wild-type plants. The height of a plant body overexpressing both AtLOG4 and CYP735A1 or CYP735A2 genes was slightly less than that of wild-type plants in week 7 after germination. However, the plant body grew continuously even in the 2^{nd} month after germination, and after that the wild-type plant had stopped the growth of their flowering stems, and thus reached a height about 1.5 times as great as that of wild-type plants.

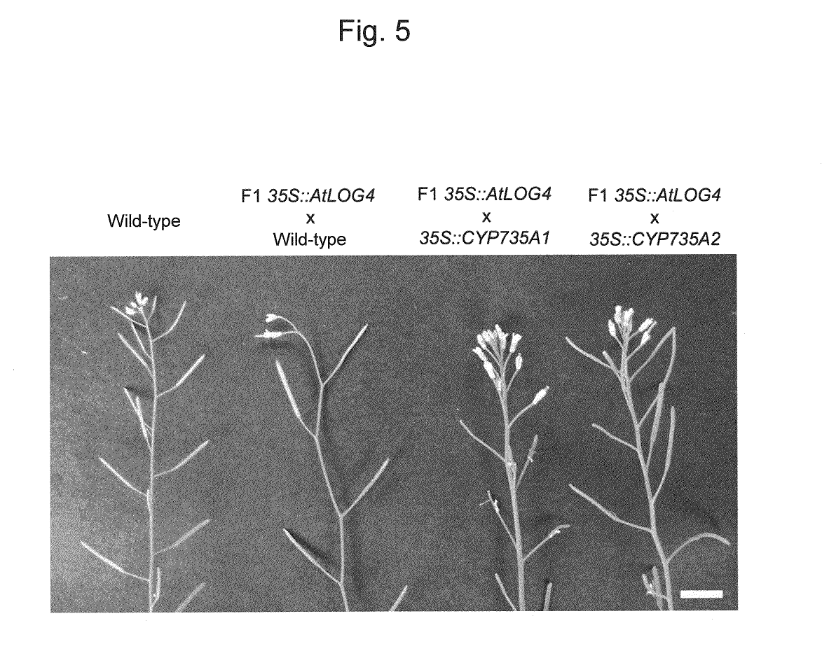

The phenotype of the inflorescences of each plant body in week 7 after germination is shown in FIG. 5. The number of inflorescences of the transgenic plants overexpressing both AtLOG4 and CYP735A1 or CYP735A2 genes was higher than that of wild-type plants or the transgenic plant overexpressing AtLOG4 alone, such that the transgenic plants overexpressing both AtLOG4 and CYP735A1 or CYP735A2 genes had many flowers that bloomed during the same period.

All publications, patents, and patent applications cited herein are incorporated herein by reference in their entirety.

### INDUSTRIAL APPLICABILITY

According to the present invention, a plant with increased biomass was produced by causing overexpression of two genes involved in cytokinin synthesis and cytokinin activity in a plant body, and by controlling the quantitative productivity of iP-type and tZ-type active cytokinins in plant aerial organs. Increased plant biomass leads to increased biofuel production, and thus biofuels can be effectively used as energy sources that are alternatives to fossil fuels.

1. A method for producing a transgenic plant with increased biomass, comprising introducing an active cytokinin synthase gene and a cytokinin hydroxylase gene into a plant cell so that they can be co-expressed, and regenerating a plant body from the plant cell.

2. The method of claim 1, wherein the active cytokinin synthase gene is a LOG gene.

3. The method of claim 1, wherein the cytokinin hydroxylase gene is a CYP735A gene.

4. The method of claim 1, wherein the active cytokinin synthase gene is any one of the following genes (a) to (f):
(a) a gene comprising DNA that consists of the nucleotide sequence shown in SEQ ID NO: 1, 3, 5, 7, 9, 11, 13, or 15;
(b) a gene comprising DNA that hybridizes under stringent conditions to DNA consisting of a nucleotide sequence complementary to DNA consisting of the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
(c) a gene comprising DNA that consists of a nucleotide sequence having 70% or higher identity with the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
(d) a gene encoding a protein that consists of the amino acid sequence shown in SEQ ID NO: 2, 4, 6, 8, 10, 12, 14, or 16;
(e) a gene encoding a protein that consists of an amino acid sequence derived from the amino acid sequence shown in SEQ ID NO: 2, 4, 6, 8, 10, 12, 14 or 16 by deletion, substitution, or addition of 1 or several amino acids, and has activity to catalyze a reaction for synthesizing an active cytokinin from a nucleotide cytokinin; and
(f) a gene encoding a protein that consists of an amino acid sequence having 70% or higher identity with the amino acid sequence shown in SEQ ID NO: 2, 4, 6, 8, 10, 12, 14, or 16, and has activity to catalyze a reaction for synthesizing an active cytokinin from a nucleotide cytokinin.

5. The method of claim 1, wherein the cytokinin hydroxylase gene is any one of the following genes (g) to (l):
(g) a gene comprising DNA that consists of the nucleotide sequence shown in SEQ ID NO: 17, 19, 21, or 23;
(h) a gene comprising DNA that hybridizes under stringent conditions to DNA consisting of a nucleotide sequence complementary to DNA consisting of the nucleotide sequence shown in SEQ ID NO: 17, 19, 21, or 23, and encodes a protein having activity to catalyze conversion from an iP-type cytokinin to a tZ-type cytokinin;
(i) a gene comprising DNA that consists of a nucleotide sequence having 70% or higher identity with the nucleotide sequence shown in SEQ ID NO: 17, 19, 21, or 23, and encodes a protein having activity to catalyze conversion from an iP-type cytokinin to a tZ-type cytokinin;
(j) a gene encoding a protein that consists of the amino acid sequence shown in SEQ ID NO: 18, 20, 22, or 24;
(k) a gene encoding a protein that consists of an amino acid sequence derived from the amino acid sequence shown in SEQ ID NO: 18, 20, 22 or 24 by deletion, substitution, or addition of 1 or several amino acids, and has activity to catalyze conversion from an iP-type cytokinin to a tZ-type cytokinin; and
(l) a gene encoding a protein that consists of an amino acid sequence having 70% or higher identity with the amino acid sequence shown in SEQ ID NO: 18, 20, 22, or 24, and has activity to catalyze conversion from an iP-type cytokinin to a tZ-type cytokinin.

6. A method for increasing plant biomass, comprising overexpressing an active cytokinin synthase gene and a cytokinin hydroxylase gene in a plant body.

7. The method of claim 6, wherein the active cytokinin synthase gene is a LOG gene.

8. The method of claim 6, wherein the cytokinin hydroxylase gene is a CYP735A gene.

9. The method of claim 6, wherein the active cytokinin synthase gene is any one of the following genes (a) to (f):
(a) a gene comprising DNA that consists of the nucleotide sequence shown in SEQ ID NO: 1, 3, 5, 7, 9, 11, 13, or 15;
(b) a gene comprising DNA that hybridizes under stringent conditions to DNA consisting of a nucleotide sequence complementary to DNA consisting of the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
(c) a gene comprising DNA that consists of a nucleotide sequence having 70% or higher identity with the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
(d) a gene encoding a protein that consists of the amino acid sequence shown in SEQ ID. NO: 2, 4, 6, 8, 10, 12, 14, or 16;
(e) a gene encoding a protein that consists of an amino acid sequence derived from the amino acid sequence shown in SEQ ID NO: 2, 4, 6, 8, 10, 12, 14 or 16 by deletion, substitution, or addition of 1 or several amino acids, and has activity to catalyze a reaction for synthesizing an active cytokinin from a nucleotide cytokinin; and
(f) a gene encoding a protein that consists of an amino acid sequence having 70% or higher identity with the amino acid sequence shown in SEQ ID NO: 2, 4, 6, 8, 10, 12, 14, or 16, and has activity to catalyze a reaction for synthesizing an active cytokinin from a nucleotide cytokinin.

10. The method of claim 6, wherein the cytokinin hydroxylase gene is any one of the following genes (g) to (l):
(g) a gene comprising DNA that consists of the nucleotide sequence shown in SEQ ID NO: 17, 19, 21, or 23;
(h) a gene comprising DNA that hybridizes under stringent conditions to DNA consisting of a nucleotide sequence complementary to DNA consisting of the nucleotide sequence shown in SEQ ID NO: 17, 19, 21 or 23, and encodes a protein having activity to catalyze conversion from an iP-type cytokinin to a tZ-type cytokinin;
(i) a gene comprising DNA that consists of a nucleotide sequence having 70% or higher identity with the nucleotide sequence shown in SEQ ID NO: 17, 19, 21 or 23, and encodes a protein having activity to catalyze conversion from an iP-type cytokinin to a tZ-type cytokinin;
(j) a gene encoding a protein that consists of the amino acid sequence shown in SEQ ID NO: 18, 20, 22, or 24;
(k) a gene encoding a protein that consists of an amino acid sequence derived from the amino acid sequence shown in SEQ ID NO: 18, 20, 22 or 24 by deletion, substitution, or addition of 1 or several amino acids, and has activity to catalyze conversion from an iP-type cytokinin to a tZ-type cytokinin; and
(l) a gene encoding a protein that consists of an amino acid sequence having 70% or higher identity with the amino acid sequence shown in SEQ ID NO: 18, 20, 22, or 24, and has activity to catalyze conversion from an iP-type cytokinin to a tZ-type cytokinin.

11. A transgenic plant, wherein an active cytokinin synthase gene and a cytokinin hydroxylase gene are introduced to increase biomass.

12. The transgenic plant of claim 11, wherein the active cytokinin synthase gene is a LOG gene.

13. The transgenic plant of claim 11, wherein the cytokinin hydroxylase gene is a CYP735A gene.

14. The transgenic plant of claim 11, wherein the active cytokinin synthase gene is any one of the following genes (a) to (f):
(a) a gene comprising DNA that consists of the nucleotide sequence shown in SEQ ID NO: 1, 3, 5, 7, 9, 11, 13, or 15;
(b) a gene comprising DNA that hybridizes under stringent conditions to DNA consisting of a nucleotide sequence complementary to DNA consisting of the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
(c) a gene comprising DNA that consists of a nucleotide sequence having 70% or higher identity with the nucleotide sequence shown in SEQ ID NO: 1, 3, 5, 7, 9, 11, 13, or 15, and encodes a protein having activity to catalyze a reaction for synthesizing an active cytokinin from a nucleotide cytokinin;
(d) a gene encoding a protein that consists of the amino acid sequence shown in SEQ ID NO: 2, 4, 6, 8, 10, 12, 14 or 16;
(e) a gene encoding a protein that consists of an amino acid sequence derived from the amino acid sequence shown in SEQ ID NO: 2, 4, 6, 8, 10, 12, 14 or 16 by deletion, substitution, or addition of 1 or several amino acids, and has activity to catalyze a reaction for synthesizing an active cytokinin from a nucleotide cytokinin; and
(f) a gene encoding a protein that consists of an amino acid sequence having 70% or higher identity with the amino acid sequence shown in SEQ ID NO: 2, 4, 6, 8, 10, 12, 14, or 16, and has activity to catalyze a reaction for synthesizing an active cytokinin from a nucleotide cytokinin.

15. The transgenic plant of claim 11, wherein the cytokinin hydroxylase gene is any one of the following genes (g) to (l):
(g) a gene comprising DNA that consists of the nucleotide sequence shown in SEQ ID NO: 17, 19, 21, or 23;
(h) a gene comprising DNA that hybridizes under stringent conditions to DNA consisting of a nucleotide sequence complementary to DNA consisting of the nucleotide sequence shown in SEQ ID NO: 17, 19, 21, or 23, and encodes a protein having activity to catalyze conversion from an iP-type cytokinin to a tZ-type cytokinin;
(i) a gene comprising DNA that consists of a nucleotide sequence having 70% or higher identity with the nucleotide sequence shown in SEQ ID NO: 17, 19, 21, or 23, and encodes a protein having activity to catalyze conversion from an iP-type cytokinin to a tZ-type cytokinin;
(j) a gene encoding a protein that consists of the amino acid sequence shown in SEQ ID NO: 18, 20, 22, or 24;
(k) a gene encoding a protein that consists of an amino acid sequence derived from the amino acid sequence shown in SEQ ID NO: 18, 20, 22 or 24 by deletion, substitution, or addition of 1 or several amino acids, and has activity to catalyze conversion from an iP-type cytokinin to a tZ-type cytokinin; and
(l) a gene encoding a protein that consists of an amino acid sequence having 70% or higher identity with the amino acid sequence shown in SEQ ID NO: 18, 20, 22, or 24, and has activity to catalyze conversion from an iP-type cytokinin to a tZ-type cytokinin.
